(19) United States
(12) Patent Application Publication
(10) Pub. No.: US 2025/0043250 A1
(43) Pub. Date: Feb. 6, 2025

(54) COMPOSITIONS AND METHODS FOR EXPANDING KERATINOCYTES

(71) Applicant: STEMCELL Technologies Canada Inc., Vancouver (CA)

(72) Inventors: Yaoyao Chen, Vancouver (CA); Wing Chang, Vancouver (CA); Victoria Stephenson, Vancouver (CA)

(21) Appl. No.: 18/718,866

(22) PCT Filed: Dec. 15, 2022
(86) PCT No.: PCT/CA2022/051834

§ 371 Date: Jun. 12, 2024

Related U.S. Application Data
(60) Provisional application No. 63/290,480, filed on Dec. 16, 2021.

(51) Int. Cl. C12N 5/071

(52) U.S. Cl. CPC C12N 5/0698; C12N 5/0627; C12N 2501/15; C12N 2501/727; C12N 2506/45

(57) ABSTRACT

This disclosure relates to media compositions and/or supplements to be added into a medium, and to methods for culturing and/or expanding keratinocytes or keratinocyte-like cells. In another aspect, this disclosure relates to media composition and/or supplements to be added into a medium, and to methods of providing an enriched or selected for population of keratinocytes or keratinocyte-like cells. Keratinocytes or keratinocyte-like cells may be derived from pluripotent stem cells, and more particularly from a differentiated population of pluripotent stem cells.

### CROSS-REFERENCE TO RELATED APPLICATIONS

This application claims the benefit of priority of U.S. Provisional Patent Application No. 63/290,480, filed Dec. 16, 2021, the entire content of which is hereby incorporated by reference in its entirety.

### TECHNICAL FIELD

This disclosure relates to cell culture applications, and more specifically to the culture and/or expansion of keratinocytes, and still more specifically to the culture and/or expansion of epidermal keratinocytes.

### BACKGROUND

In addition to their use as a research tool, the ability to expand keratinocytes, such as epidermal keratinocytes, would have important medical applications. An expandable and potentially limitless source of keratinocytes would be of immense importance to victims of burns, and others in need of skin grafts.

In addition, an expandable and potentially limitless source of keratinocytes, such as epidermal keratinocytes, would be a valuable resource to industries, such as the cosmetics industry, for testing the effects of products and product-components in an in vitro model system.

Still further, an expandable and potentially limitless source of keratinocytes, such as epidermal keratinocytes, would enable scientific research that studies developmental biology of the skin and diseases related to the skin, such as melanoma, and other skin cancers.

However, primary skin samples are scarce and primary human keratinocyte culture has a finite lifespan. Thus, the development of media and methods for deriving keratinocytes from pluripotent stem cells (PSCs) would provide research and industrial laboratories, and potentially the medical field, with an indefinite source of experimental or therapeutic material. Indeed, PSC-derived keratinocytes will enable the modeling of skin diseases, whether inherited or spontaneous, and provide hope for patient-specific cell therapy in the clinic.

Therefore, there is a need for robust and efficient media, kits, methods and systems to generate and expand populations of PSC-derived keratinocytes, such as PSC-derived epidermal keratinocytes.

### SUMMARY

In one aspect of this disclosure are provided methods for generating and/or expanding and/or enriching keratinocytes, such as epidermal keratinocytes. The methods may comprise, contacting a differentiated population of pluripotent stem cells (PSCs) with an expansion medium comprising a basal medium and one or more of an inhibitor of transformation growth factor (TGF) signaling, a gamma secretase inhibitor, and an agent that disrupts cytoskeletal structure, and culturing the differentiated population of PSCs in the expansion medium to generate expanded keratinocytes.

In one embodiment, the expansion medium is supplemented with two or more of the inhibitor of TGF signaling, the gamma secretase inhibitor, and the agent that disrupts cytoskeletal structure.

In one embodiment, the expansion medium is serum- and/or bovine pituitary extract-free.

In one embodiment, the contacting and the culturing steps are in feeder cell free conditions.

In one embodiment, the contacting and the culturing steps are performed on or in a support comprising one or more extracellular matrix proteins.

In one embodiment, the methods may further comprise dissociating a PSC-derived skin organoid to obtain the differentiated population of PSCs. In one embodiment, the PSC-derived skin organoids are formed under serum- and or feeder cell-free conditions.

In one embodiment, the PSC-derived skin organoid is a PSC-derived hair-bearing skin organoid.

In one embodiment, the methods may further comprise yielding more epidermal keratinocytes than contaminating cell types when the contacting and the culturing steps are performed in the expansion medium compared to the basal medium not supplemented with one or more of the inhibitor of TGFB signaling, the gamma secretase inhibitor, and the agent that disrupts cytoskeletal structure.

In one embodiment, wherein the inhibitor of TGF signaling is an inhibitor of TGF-beta signaling. In one embodiment, the inhibitor of TGF-beta signaling is one or more of A83-01, A77-01, and SB431542.

In one embodiment, the gamma secretase inhibitor is DAPT.

In one embodiment, the agent that disrupts cytoskeletal structure is a Rho/Rock kinase inhibitor. In one embodiment, the Rho/Rock kinase inhibitor is Y-27632.

In another aspect of this disclosure are provided media for generating and/or expanding and/or enriching keratinocytes, such as epidermal keratinocytes. The media may comprise a basal medium supplemented with one or more of an inhibitor of transformation growth factor (TGF) signaling, a gamma secretase inhibitor, and an agent that disrupts cytoskeletal structure (e.g. an expansion medium).

In one embodiment, the inhibitor of TGF signaling is an inhibitor of TGF-beta signaling. In one embodiment, the inhibitor of TGF-beta signaling is one or more of A83-01, A77-01, and SB431542.

In one embodiment, the gamma secretase inhibitor is DAPT.

In one embodiment, the agent that disrupts cytoskeletal structure is a Rho/Rock kinase inhibitor. In one embodiment, the Rho/Rock kinase inhibitor is Y-27632.

In one embodiment, the medium does not come into contact with feeder cells to expand the (epidermal) keratinocytes.

In one embodiment, the (epidermal) keratinocytes are derived from a differentiated population of PSCs.

In one embodiment, the medium is serum- and/or BPE-free.

In one embodiment, the medium is free of an exogenously added inhibitor of TGF signaling.

In one embodiment, the medium is free of an exogenously added gamma secretase inhibitor.

In one embodiment, the medium supports the expansion of epithelial cells.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows representative photographs of human cells in culture. Human primary neonatal keratinocytes (HEKn3) were cultured as a monolayer in DermaCult™ (STEMCELL Technologies) medium and imaged on day 25 of the third passage (A). Human PSC were cultured as a monolayer using a published protocol (Kajiwara K, et al, 2017) in Defined Keratinocyte Serum Free Medium (ThermoFisher) (B). Scale bar is 200 μm.

FIG. 2 shows representative images corresponding to different stages of a keratinocyte differentiation protocol of this disclosure using WLS-1C iPS cells. In comparison to the results shown in FIG. 1, the efficiency of keratinocyte derivation from PSC could be improved when the cells of hair-bearing skin organoids are used as the starting point. Panel A) shows a day 70-120 PSC-derived hair-bearing skin organoid. The images in panels B)-D) correspond to days 4, 7 and 10 after the dissociated cells of PSC-derived hair-bearing skin organoids are plated in a monolayer in a culture medium of this disclosure. Scale bar is 200 μm.

FIG. 3 shows a line graph of keratinocyte population doublings. During extended culture in a culture medium of this disclosure the population doublings of H9 ES cells, H1 ES cells, and STiPS-F022 iPS cells were compared to primary neonatal keratinocytes and to primary adult keratinocytes.

FIG. 4 shows the effects of an extracellular matrix coating on the expansion of keratinocytes. Differentiated STiPS-F022 iPS cells were plated as a monolayer, either directly on plastic dishes or on a collagen coating, in contact with a culture medium of this disclosure, and the cells were grown for at least 6 passages (p6). Images were taken in each condition on day 5 after plating and on day 5 of the sixth passage (A). Expansion of STiPS-F022 iPS-derived keratinocytes was assessed among cells plated either directly on plastic dishes or on a collagen coating (B). Scale bar is 200 μm.

FIG. 5 shows immunocytochemistry staining for the expression of keratinocyte-specific markers among keratinocytes derived/expanded from PSC. WLS-1C iPS cells were differentiated into keratinocyte-like cells in accordance with this disclosure and assessed for Keratin 14 (K14), Keratin 5 (K5), and TP63 (p63) expression at either passage 1 (A) or passage 9 (B). H9 ES cells were differentiated into keratinocyte-like cells in accordance with this disclosure and assessed for K14, K5, and p63 expression at passage 4 (C). Expression levels of K14, K5, and p63 among PSC-derived keratinocytes was assessed against a control culture of human primary keratinocytes (D). Scale bar is 200 μm.

FIG. 6 shows the morphology of colonies of keratinocytes seeded at a clonal cell density. Both primary neonatal keratinocytes (HEKn3) and the cells of dissociated PSC-derived skin organoids (STiPS-F022) were seeded at 200 cells/well of a 6-well plate and cultured in DermaCult™ but in the absence of a coating or feeder cell support. The image shows K14+ cells/colonies in grey.

FIG. 7 shows the morphology of PSC-derived cells after culturing in various commercially available media. STiPS-F022 iPS cells were formed into hair-bearing skin organoids as described herein and dissociated prior to plating the cells in either EpiLife™ EDGS medium (ThermoFisher) (A), EpiLife™ HKGS medium (ThermoFisher) (B), or CnT-57 medium (CELLnTEC) (C). Images of cells growing in each medium condition were taken at the indicated time points. Scale bar is 200 μm.

FIG. 8 shows a line graph of population doublings of PSC-derived cells grown in various culture media. H9 ES cells were grown in either a keratinocyte differentiation medium of this disclosure or in EpiLife HKGS medium, and population doublings were assessed over numerous weeks. STiPS-F022 iPS cells were grown in either a keratinocyte differentiation medium of this disclosure or in EpiLife EDGS medium and population doublings were assessed over numerous weeks. H1 ES cells were grown in a keratinocyte differentiation medium of this disclosure and population doublings were assessed over numerous weeks.

FIG. 9 shows the expression of keratin 14 among PSC-derived cells cultured in different media formulations. Representative immunocytochemistry images of keratin 14 staining among cells derived from H1 and H9 ES cells and WLS-1C and STiPS-F022 iPS cells using either a medium of this disclosure, EpiLife HKGS, EpiLife EDGS, or CnT57 culture medium (A). The plot shows the percentage of keratin 14 positive cells differentiated from the indicated PSC lines and in the indicated culture media (B). Scale bar is 200 μm.

FIG. 10 shows the expression of TP63 among PSC-derived cells cultured in different media formulations. Representative immunocytochemistry images of TP63 staining among cells derived from H1 and H9 ES cells and WLS-1C and STiPS-F022 iPS cells using either a medium of the disclosure, EpiLife HKGS, EpiLife EDGS, or CnT57 culture medium (A). The plots show the percentage of TP63 positive cells among keratinocytes differentiated from the indicated PSC lines and in the indicated culture media (B). Scale bar is 200 μm.

FIG. 11 shows the morphology of PSC-derived cells after culturing in various media formulations. H9 ES cells and WLS-1C iPS cells were formed into hair-bearing skin organoids as described herein and dissociated prior to plating the cells in DermaCult, EpiLife HKGS, or in EpiLife HKGS supplemented with different small molecules. Images of cells growing in each medium condition were taken at the indicated time points. Scale bar is 200 μm.

FIG. 12 shows a FACS analysis of TP63+ cells generated in various media formulations. WLS-1C iPS cells were formed into hair-bearing skin organoids as described herein and dissociated prior to plating the cells in EpiLife HKGS, or in EpiLife HKGS supplemented with the indicated small molecules. The cells were stained for TP63 expression and analyzed by FACS.

### DETAILED DESCRIPTION

This disclosure relates to media compositions and/or supplements to be added into a medium, and to methods for culturing human keratinocytes. More specifically, this disclosure relates to the expansion of or the expansion conditions for human keratinocytes rather than the expansion (conditions) for off-target or contaminating cell-types present in a starting population of cells. In one embodiment, this disclosure relates to the preferential or selective expansion of keratinocytes (e.g. epidermal keratinocytes) in comparison to other cell types found in the skin, such as fibroblasts.

Where used in this disclosure, the term “keratinocytes” refers to a cell type that is found in various animals, and particularly within the skin thereof, but also in the esophagus, for example. In humans and other animals, keratinocytes make up the majority of the outer layer of the skin (i.e. the epidermis). Thus, in embodiments of this disclosure the term “keratinocytes” refers to epidermal keratinocytes, such as human epidermal keratinocytes. As such, the terms “keratinocytes” and “epidermal keratinocytes” are interchangeable herein. In the context of skin, keratinocytes differentiate from an underlying basal layer and migrate upward toward the surface of the epidermis. In humans, keratinocytes may make up about 90% or more of epidermal skin cells. During the development of keratinocytes from epidermal stem cells, different stages of keratinocyte development are characterized by the expression of stage-specific markers. Keratinocytes may be characterized by the expression of TP63, keratin 5 (“K5”), and keratin 14 (“K14”). In vivo, keratinocytes have at least some ability to proliferate before they differentiate and/or senesce and/or become shed.

Where used in this disclosure, the term “pluripotent stem cell” or “PSC”, or pluralized variations, refers to a population of cells that are capable of self-renewal and also capable of differentiating to all three germ layers, and beyond. The conditions for culturing PSCs to maintain them in an undifferentiated state are known, but improvements to these conditions are the subject of ongoing research. PSC is a broad term that captures both embryonic stem cells (“ESCs”) and induced pluripotent stem cells (“iPSC”), and the like. While there may be ethical challenges with sourcing ESCs, by now several ESC lines are well established in the field. On the other hand, iPSCs may be induced from a great variety of readily accessible cells, including adult somatic cells, and are therefore associated with fewer ethical constraints. Thus, new iPSC lines emerge with regularity. PSCs are an important model to study differentiation mechanisms, to model diseases, and offer significant medical opportunities. PSCs may be obtained, derived or induced from any source species, but in this disclosure PSCs are preferably human.

Where used in this disclosure, the term “differentiated population of PSCs” refers to a population of cells that at one point emerged from one or more undifferentiated PSCs, but as a result of being exposed to various internal or external cues are no longer in an undifferentiated state. While a differentiated population of PSCs may still retain some capacity to self-renew, the ability of the cells of such population to differentiate to any lineage may be constrained by the developmental trajectory taken (as influenced by the internal or external cue(s) to which they had been exposed). In the context of this disclosure, a differentiated population of PSCs is a population of cells that has been prompted toward the ectodermal lineage. More specifically, a differentiated population of PSCs is a population of cells that is further downstream of early ectoderm toward or to the epidermal lineage. Beginning from a PSC, a differentiated population of PSCs may differentiate directly to the cell type of interest or may go through one or more intermediate stages. Thus, a differentiated population of PSCs capable of giving rise to an epidermal stem cell may have gone through one or more earlier stages, including an ectodermal progenitor cell stage. In one embodiment, the differentiated population of PSCs corresponds to the cells of a dissociated PSC-derived skin organoid. PSC-derived skin organoids may be generated in any way known in the art, including as published by Lee et al. (Nature, 2020). In one embodiment, the differentiated population of PSCs corresponds to the cells of a hair-bearing PSC-derived skin organoid. In one embodiment, the differentiated population of PSCs corresponds to the cells of a dissociated hair-bearing PSC-derived skin organoid. A differentiated population of PSCs may correspond to any source species, but in this disclosure the differentiated population of PSCs are preferably human.

### Media and Supplements

In one aspect of this disclosure are provided media for expanding keratinocytes, such as epidermal keratinocytes. In one embodiment, the (epidermal) keratinocytes are primary-derived, that is to say directly obtained from a subject or patient. In one embodiment, the (epidermal) keratinocytes are PSC-derived. In one embodiment, the (epidermal) keratinocytes are derived from a differentiated population of PSCs. Thus, the differentiated population of PSCs may be intermediate of the PSCs and the (epidermal) keratinocytes.

In one embodiment, a differentiated population of PSCs (such as from a skin organoid or a hair bearing skin organoid) may comprise one or more epidermal stem cells. In one embodiment, a differentiated population of PSCs (such as from a skin organoid or a hair bearing skin organoid) may comprise one or more progenitors of keratinocytes or keratinocyte-like cells. In one embodiment, a differentiated population of PSCs (such as from a skin organoid or a hair bearing skin organoid) may comprise both one or more epidermal stem cells and one or more progenitors of keratinocytes or keratinocyte-like cells.

In one embodiment, the (epidermal) keratinocytes or keratinocyte-like cells are mammalian. In one embodiment, the (epidermal) keratinocytes or keratinocyte-like cells are human. In one embodiment, the keratinocytes are PSC-derived. In one embodiment, the keratinocytes or keratinocyte-like cells are expandable and/or self-renewing. Thus, in one embodiment, the keratinocytes may be or may comprise stem cells and/or keratinocyte progenitor cells.

In one embodiment, a differentiated population of PSCs (such as from a skin organoid or a hair bearing skin organoid) may comprise one or more holoclones. In one embodiment, a differentiated population of PSCs (such as from a skin organoid or a hair bearing skin organoid) may comprise one or more meroclones. In one embodiment, a differentiated population of PSCs (such as from a skin organoid or a hair bearing skin organoid) may comprise one or more paraclones. In one embodiment, a differentiated population of PSCs (such as from a skin organoid or a hair bearing skin organoid) may comprise more than one holoclones and more than one meroclones. In one embodiment, a differentiated population of PSCs (such as from a skin organoid or a hair bearing skin organoid) may comprise more holoclones and/or meroclones than paraclones.

A keratinocyte medium of this disclosure may be used to expand keratinocytes, such as epidermal keratinocytes and/or keratinocyte-like cells, and may thus be referred to as a keratinocyte expansion medium or an epidermal keratinocyte expansion medium. The foregoing terminology may be used interchangeably herein.

Keratinocyte media of this disclosure will comprise a basal medium. In one embodiment, the basal medium may be any medium that is capable of supporting the expansion of keratinocytes, such as epidermal keratinocytes or keratinocyte-like cells. In one embodiment, the basal medium may be any medium that is capable of supporting the expansion of PSC-derived keratinocytes, such as PSC-derived epidermal keratinocytes. Basal media are well known in the art, and may include RPMI, DMEM, F-12, MCDB153, DMEM/F-12, Adv DMEM, Adv DMEM/F-12. Basal medium typically include carbohydrates, amino acids, trace elements, lipids, buffers, salts, and the like. In some embodiments, basal media may not include one or more of the foregoing types of components, and which may be included in a supplement. In some embodiments, basal media may be further supplemented with one or more of the foregoing types of components.

A basal medium used to formulate (epidermal) keratinocyte expansion media of this disclosure may also be supplemented with additional components to make a complete medium. Thus, a keratinocyte medium (e.g. an epidermal keratinocyte expansion medium) of this disclosure may be formulated as a complete medium, or as a basal medium provided together (whether in a kit or otherwise) with one or more supplements to be added to the basal medium prior to use to make a complete medium.

To formulate complete (epidermal) keratinocyte expansion media, it may be beneficial and/or necessary to include (or further supplement with) one or more of: one or more growth factors or other proteins, one or more hormones, salts, vitamins, an albumin source, one or more small molecules, etc.

In one embodiment, an albumin source is included in keratinocyte expansion media of this disclosure. In one embodiment, the albumin source may be a serum, such as a serum obtained from a mammal. In one embodiment, the albumin source is a more defined component than a serum. For example, an albumin source may be an isolated albumin. In one embodiment, the isolated albumin is a recombinant albumin. In one embodiment, the isolated albumin is a bovine serum albumin. In one embodiment, the isolated albumin is a human albumin.

In one embodiment, keratinocyte media (e.g. keratinocyte expansion media or epidermal keratinocyte expansion media) of this disclosure are serum-free.

In one embodiment, keratinocyte expansion media benefit from and/or require supplementation with one or more small molecules. In one embodiment, the one or more small molecules include one or more small molecule inhibitors. In one embodiment, the one or more small molecules (e.g. small molecule inhibitors) are comprised in a supplement.

In one embodiment, keratinocyte media (e.g. keratinocyte expansion media or epidermal keratinocyte expansion media) comprise a basal medium supplemented with one or more of an inhibitor of transformation growth factor (TGF) signaling, a gamma secretase inhibitor, and an agent that disrupts cytoskeletal structure.

The inhibitor of transformation growth factor (TGF) signaling may be any factor that performs the function of inhibiting signaling through a TGF. In one embodiment, an inhibitor of transformation growth factor (TGF) signaling is a small molecule, such as a small molecule inhibitor. In one embodiment, an inhibitor of transformation growth factor (TGF) signaling is a protein. In one embodiment, an inhibitor of TGF signaling is an inhibitor of TGF-beta signaling. In one embodiment, the inhibitor of TGF signaling is one or more of A83-01, A77-01, and SB431542, Galunisertib (LY2157299), LY2109761, SB525334, SB505124, GW788388, LY364947.

The gamma secretase inhibitor may be any factor that performs the function of inhibiting signaling through a gamma secretase. In one embodiment, a gamma secretase inhibitor is a small molecule, such as a small molecule inhibitor. In one embodiment, a gamma secretase inhibitor is a protein. In one embodiment, a gamma secretase inhibitor is one or more of DAPT, RO4929097, Semagacestat (LY450139), Avagacestat (BMS-708163), Dibenzazepine (YO-01027), LY411575, L-685,458, Crenigacestat (LY3039478).

The agent that disrupts cytoskeletal structure may be any factor that performs the function of disrupting the cytoskeleton. In one embodiment, an agent that disrupts cytoskeletal structure is a small molecule, such as a small molecule inhibitor. In one embodiment, an agent that disrupts cytoskeletal structure is a protein. In one embodiment, an agent that disrupts cytoskeletal structure is a Rho/Rock kinase inhibitor. In one embodiment, an agent that disrupts cytoskeletal structure is one or more of Y-27632, Thiazovivin, Fasudil (HA-1077), GSK429286A, RKI-1447, Y-27632, H-1152 dihydrochloride, Azaindole 1 (TC-S 7001).

In one embodiment, keratinocyte media (e.g. keratinocyte expansion media or epidermal keratinocyte expansion media) comprise a basal medium supplemented with two or more of an inhibitor of transformation growth factor (TGF) signaling, a gamma secretase inhibitor, and an agent that disrupts cytoskeletal structure. In one embodiment, the two or more small molecules (e.g. small molecule inhibitors) are comprised in a supplement.

In one embodiment, keratinocyte media (e.g. keratinocyte expansion media or epidermal keratinocyte expansion media) comprise a basal medium supplemented with each of an inhibitor of transformation growth factor (TGF) signaling, a gamma secretase inhibitor, and an agent that disrupts cytoskeletal structure. In one embodiment, each of the small molecules (e.g. small molecule inhibitors) are comprised in a supplement.

In one embodiment, epidermal keratinocyte expansion media of this disclosure may be defined. That is to say, in one embodiment epidermal keratinocyte expansion media of this disclosure do not include and/or come in to contact with undefined components. Examples of undefined components that are frequently included in cell culture media, or come into contact with cell culture media, include bovine pituitary extract (“BPE”), serum, human platelet lysate, feeder cells, isolated extracellular matrix proteins produced by cells (e.g. Matrigel™), conditioned media, etc.

In one embodiment, keratinocyte media (e.g. keratinocyte expansion media or epidermal keratinocyte expansion media) of this disclosure are serum- and/or BPE-free. In one embodiment, media of this disclosure do not come into contact with feeder cells or undefined cell supports (e.g. matrices), such as Matrigel™.

In one embodiment, keratinocytes (e.g. epidermal keratinocytes) expanding or expanded in the presence of a medium of this disclosure may be cultured in the presence of an extracellular matrix, and preferably in the presence of a defined extracellular matrix coating, such as on a collagen coating.

Thus, keratinocyte media (e.g. keratinocyte expansion media or epidermal keratinocyte expansion media) of this disclosure, whether provided as a complete medium or as a basal medium to be supplemented as described herein, will support the expansion of (epidermal) keratinocytes, whether PSC-derived or primary. In one embodiment, media of this disclosure will support 5 or more, 10 or more, 15 or more, 20 or more, 25 or more, 30 or more, 35 or more, 40 or more, 45 or more, 50 or more, 55 or more, or 60 or more keratinocyte population doublings.

In one embodiment, keratinocytes, such as epidermal keratinocytes, expanded using media of this disclosure express characteristic markers. In one embodiment, more than 40% of the expanded cells are positive for markers characteristic of (epidermal) keratinocytes. In one embodiment, more than 50% of the expanded cells are positive for markers characteristic of (epidermal) keratinocytes. In one embodiment, more than 60% of the expanded cells are positive for markers characteristic of (epidermal) keratinocytes. In one embodiment, more than 70% of the expanded cells are positive for markers characteristic of (epidermal) keratinocytes. In one embodiment, more than 80% of the expanded cells are positive for markers characteristic of (epidermal) keratinocytes. In one embodiment, more than 90% of the expanded cells are positive for markers characteristic of (epidermal) keratinocytes. In one embodiment, nearly 100% of the expanded cells are positive for markers characteristic of (epidermal) keratinocytes.

In one embodiment, markers characteristic of keratinocytes, such as epidermal keratinocytes, include one or more of K14 and p63. In one embodiment, more than 40% of the expanded cells express one or both of K14 and p63 by the second passage, the third passage, the fourth passage, or the fifth passage. In one embodiment, more than 50% of the expanded cells express one or both of K14 and p63 by the second passage, the third passage, the fourth passage, or the fifth passage. In one embodiment, more than 60% of the expanded cells express one or both of K14 and p63 by the second passage, the third passage, the fourth passage, or the fifth passage. In one embodiment, more than 70% of the expanded cells express one or both of K14 and p63 by the second passage, the third passage, the fourth passage, or the fifth passage. In one embodiment, more than 80% of the expanded cells express one or both of K14 and p63 by the second passage, the third passage, the fourth passage, or the fifth passage. In one embodiment, more than 90% of the expanded cells express one or both of K14 and p63 by the second passage, the third passage, the fourth passage, or the fifth passage. In one embodiment, nearly 100% of the expanded cells express one or both of K14 and p63 by the second passage, the third passage, the fourth passage, or the fifth passage.

In one embodiment, keratinocyte media (e.g. keratinocyte expansion media or epidermal keratinocyte expansion media) of this disclosure outperform (in terms of the proportion of output cells that are actually keratinocytes) commercially available media. For example, media promoted for the purpose of expanding primary keratinocytes include EpiLife HKGS, EpiLife EDGS, KSFM, DKSFM, KGM2, KGM gold, CnT-07, CnT-57 and CnT-Prime. In addition, various of these media products may have other limitations, such as not being defined.

Thus, keratinocyte media of this disclosure may support more population doublings than the foregoing media formulations. In addition or in the alternative, epidermal keratinocyte expansion media of this disclosure may produce a greater proportion of expanded keratinocytes, such as epidermal keratinocytes, than the foregoing media formulations.

In one embodiment, keratinocyte media (e.g. keratinocyte expansion media or epidermal keratinocyte expansion media) of this disclosure preferentially or selectively (in comparison to other media) support the growth of keratinocytes, such as epidermal keratinocytes, whether PSC-derived or sourced from a primary tissue sample. In such embodiment, off-target cells may be out-competed by the target cells (e.g. keratinocytes) when cultured in the presence of keratinocyte media (e.g. keratinocyte expansion media or epidermal keratinocyte expansion media) of this disclosure. In contrast, target cells (e.g. keratinocytes) may be out-competed by the off-target cells (e.g. fibroblasts, non-keratinocyte epidermal cells) when cultured in the presence of currently available media, such as those commercial media listed in preceding paragraphs.

### Methods

In one aspect of this disclosure are provided methods for expanding keratinocytes, such as epidermal keratinocytes. In one embodiment, the (epidermal) keratinocytes are primary-derived, that is to say directly obtained from a subject or patient. In one embodiment, the (epidermal) keratinocytes are PSC-derived. In one embodiment, the (epidermal) keratinocytes are derived from a differentiated population of PSCs. Thus, the differentiated population of PSCs may be intermediate of the PSCs and the (epidermal) keratinocytes.

In one embodiment, the (epidermal) keratinocytes or keratinocyte-like cells are mammalian. In one embodiment, the (epidermal) keratinocytes or keratinocyte-like cells are human. In one embodiment, the mammalian keratinocytes are PSC-derived. In one embodiment, the keratinocytes or keratinocyte-like cells are expandable and/or self-renewing. Thus, in one embodiment, the keratinocytes may be or may comprise stem cells and/or keratinocyte progenitor cells.

In one embodiment, the differentiated population of PSCs may arise from or correspond to the cells of a skin organoid (e.g. a PSC-derived skin organoid). In one embodiment, the dissociated skin organoid may correspond to the cells of a hair-bearing skin organoid. In one embodiment, the differentiated population of PSCs may be a dissociated population of cells derived from a skin organoid, such as hair-bearing skin organoid.

The description of keratinocyte media (e.g. keratinocyte expansion media or epidermal keratinocyte expansion media) provided above applies to any reference to such media in the context of methods, kits, and uses described herein.

A method of expanding keratinocytes, such as epidermal keratinocytes, may comprise contacting a differentiated population of pluripotent stem cells (PSCs) with a keratinocyte medium (as described above), and culturing the differentiated population of PSCs in such medium to generate expanded keratinocytes, such as epidermal keratinocytes.

In another aspect, a method of providing an enriched or selected for population of keratinocytes, such as epidermal keratinocytes, comprises contacting a differentiated population of pluripotent stem cells (PSCs) with a keratinocyte medium, and culturing the differentiated population of PSCs in such medium to generate enriched and/or expanded keratinocytes, such as epidermal keratinocytes. An enriched population of cells refers to the scenario where target cells, in this case keratinocytes, predominate over each population, whether alone or combined, of off-target cells (such as fibroblasts, non-keratinocyte epidermal cells, etc).

In one embodiment, a differentiated population of PSCs (such as from a skin organoid or a hair bearing skin organoid) may comprise one or more epidermal stem cells. In one embodiment, a differentiated population of PSCs (such as from a skin organoid or a hair bearing skin organoid) may comprise one or more progenitors of keratinocytes or keratinocyte-like cells. In one embodiment, a differentiated population of PSCs (such as from a skin organoid or a hair bearing skin organoid) may comprise both one or more epidermal stem cells and one or more progenitors of keratinocytes or keratinocyte-like cells.

In one embodiment, a differentiated population of PSCs (such as from a skin organoid or a hair bearing skin organoid) may comprise one or more holoclones. In one embodiment, a differentiated population of PSCs (such as from a skin organoid or a hair bearing skin organoid) may comprise one or more meroclones. In one embodiment, a differentiated population of PSCs (such as from a skin organoid or a hair bearing skin organoid) may comprise one or more paraclones. In one embodiment, a differentiated population of PSCs (such as from a skin organoid or a hair bearing skin organoid) may comprise more than one holoclones and more than one meroclones. In one embodiment, a differentiated population of PSCs (such as from a skin organoid or a hair bearing skin organoid) may comprise more holoclones and/or meroclones than paraclones.

The contacting and culturing steps may be for any duration of time that may yield a desired quantity of enriched and/or expanded keratinocytes, such as epidermal keratinocytes. In one embodiment, the contacting and culturing steps are carried out for about 1 day, about 2 days, about 3 days, about 4 days, about 5 days, about 6 days, about 7 days, about 8 days, about 9 days, about 10 days, about 11 days, about 12 days, about 13 days, or about 14 days.

In one embodiment, the contacting and culturing steps are carried out for more than one, more than two, more than three, more than four, more than five, more than six, more than seven, more than eight, more than nine, or more than ten passages. Naturally, the expanded or expanding population of (epidermal) keratinocytes may be briefly removed from contact with a keratinocyte medium for the period of time when the culture is being passaged.

In one embodiment, the contacting and culturing steps are in feeder cell free conditions. In one embodiment, the contacting and the culturing steps are performed on or in a support or a coating comprising one or more extracellular matrix proteins. In one embodiment, the support or coating comprising one or more extracellular matrix proteins is undefined, such as Matrigel™. In one embodiment, the support or coating comprising one or more extracellular matrix proteins is defined, such as a collagen, a vitronectin, a laminin, or the like. In one embodiment, the one or more extracellular matrix proteins are recombinant. In one embodiment, the contacting and the culturing steps are performed in the absence of a support or a coating comprising one or more extracellular matrix proteins.

As described above, expansion media of this disclosure comprise a basal medium supplemented with one or more of an inhibitor of transformation growth factor (TGF) signaling, a gamma secretase inhibitor, and an agent that disrupts cytoskeletal structure.

The inhibitor of transformation growth factor (TGF) signaling may be any factor that performs the function of inhibiting signaling through a TGF. In one embodiment, an inhibitor of transformation growth factor (TGF) signaling is a small molecule, such as a small molecule inhibitor. In one embodiment, an inhibitor of transformation growth factor (TGF) signaling is a protein. In one embodiment, an inhibitor of TGF signaling is an inhibitor of TGF-beta signaling. In one embodiment, the inhibitor of TGF signaling is one or more of A83-01, A77-01, and SB431542, Galunisertib (LY2157299), LY2109761, SB525334, SB505124, GW788388, LY364947.

The gamma secretase inhibitor may be any factor that performs the function of inhibiting signaling through a gamma secretase. In one embodiment, a gamma secretase inhibitor is a small molecule, such as a small molecule inhibitor. In one embodiment, a gamma secretase inhibitor is a protein. In one embodiment, a gamma secretase inhibitor is one or more of DAPT, RO4929097, Semagacestat (LY450139), Avagacestat (BMS-708163), Dibenzazepine (YO-01027), LY411575, L-685,458, Crenigacestat (LY3039478).

The agent that disrupts cytoskeletal structure may be any factor that performs the function of disrupting the cytoskeleton. In one embodiment, an agent that disrupts cytoskeletal structure is a small molecule, such as a small molecule inhibitor. In one embodiment, an agent that disrupts cytoskeletal structure is a protein. In one embodiment, an agent that disrupts cytoskeletal structure is a Rho/Rock kinase inhibitor. In one embodiment, an agent that disrupts cytoskeletal structure is one or more of Y-27632, Thiazovivin, Fasudil (HA-1077), GSK429286A, RKI-1447, Y-27632, H-1152 dihydrochloride, Azaindole 1 (TC-S 7001).

In one embodiment, keratinocyte media (e.g. keratinocyte expansion media or epidermal keratinocyte expansion media) of this disclosure comprise a basal medium supplemented with two or more of an inhibitor of transformation growth factor (TGF) signaling, a gamma secretase inhibitor, and an agent that disrupts cytoskeletal structure.

In one embodiment, keratinocyte media (e.g. keratinocyte expansion media or epidermal keratinocyte expansion media) of this disclosure comprise a basal medium supplemented with each of an inhibitor of transformation growth factor (TGF) signaling, a gamma secretase inhibitor, and an agent that disrupts cytoskeletal structure.

In one embodiment, the supplemented, as described herein, basal medium (e.g. an epidermal keratinocyte expansion medium) is serum- and/or bovine pituitary extract-free. In one embodiment, the supplemented, as described herein, basal medium (e.g. an epidermal keratinocyte expansion medium) is defined.

In one embodiment, the methods may further comprise dissociating a PSC-derived skin organoid to obtain the differentiated population of PSCs. In one embodiment, the methods may further comprise providing a dissociated sample of a differentiated population of PSCs. In one embodiment, the dissociated sample is obtained by dissociating a skin organoid. In one embodiment, the skin organoid is a hair-bearing skin organoid. In one embodiment, the skin organoid and/or the hair-bearing skin organoid is PSC-derived. In one embodiment, the skin organoid and/or the hair-bearing skin organoid is derived from human cells, such as human PSCs.

In one embodiment, the skin organoid and/or hair-bearing skin organoid is derived according to published methods. In one embodiment, the skin organoid and/or hair-bearing skin organoid is derived using the STEMdiff™ Skin Organoid Kit (STEMCELL Technologies).

In one embodiment, the skin organoid and/or hair-bearing skin organoid is formed under serum- and or feeder cell-free conditions. In one embodiment, the skin organoid and/or hair-bearing skin organoid is formed in the presence of Matrigel™. In one embodiment, the skin organoid and/or hair-bearing skin organoid is formed in the absence of Matrigel™. In one embodiment, the skin organoid and/or hair-bearing skin organoid is formed using low attachment, ultra-low attachment, or non-tissue culture treated plates (e.g. 96-well, 24-well, or 6 well, etc). In one embodiment, the plates used to form skin organoid and/or hair-bearing skin organoid, may be coated with a surfactant, such as the Anti-adherence Rinse Solution (STEMCELL Technologies) prior to seeding cells therein.

In one embodiment, the skin organoid and/or hair-bearing skin organoid is formed in a step-wise manner, and takes a minimum of 15-, 20-, 25-, 30-, 35-, 40-, 45-, 50-, 55-, 60-, 65-, 70-, 75-, 80-, 85-, 90-, 95-, 100-, 105-, 110-, 115-, 120-, 125-, 130-, 135-, 140-, 145-, 150-days, or more before the structure and cell types have developed.

In one embodiment, skin (or hair-bearing skin) organoids may be dissociated using any known dissociation means. In one embodiment, the dissociation means may be protease-based. In one embodiment, the dissociation means may be Trypsin, Dispase, TrypILE™, Accutase™.

In one embodiment, skin (or hair-bearing skin) organoids may be dissociated at various time points during their developmental timeline, and still yield expanded keratinocytes when cultured in accordance with media and methods of this disclosure. Thus, in one embodiment, skin (or hair-bearing skin) organoids (e.g. the population of differentiated PSCs) having been dissociated at various time points may nevertheless be used to obtain expanded and/or enriched keratinocytes (e.g. epidermal keratinocytes). Without being bound by theory, an appropriate progenitor of keratinocytes (e.g. epidermal keratinocytes) may be present before the skin (or hair-bearing skin) organoid fully develops and may persist through to such later stage of development. In one embodiment, day 70, day 100, or day 140 organoids may be used to derive expanded and/or enriched (epidermal) keratinocytes in a medium (or practicing the methods) of this disclosure.

The methods (and use of the media) of this disclosure yield expanded epidermal keratinocytes. The expanded epidermal keratinocytes should exhibit cobblestone and polygonal epithelial cell morphology. Further, there should be few or no elongated or spindled cells, which may resemble fibroblasts and/or other contaminating, off-target cell types. In one embodiment, a quantity of fibroblasts and/or other contaminating or off-target cells decreases during a passage or following a passage, while a quantity of (epidermal) keratinocytes or keratinocyte-like cells increases during the passage or following the passage.

In one embodiment, the expanded keratinocytes grow as colonies. In one embodiment, the expanded keratinocytes grow as clonally-derive colonies. In one embodiment, the colonies are derived from a holoclone or a holoclone-like cell (in the case of PSC-derived cultured). In one embodiment, the colonies are derived from a meroclone or a meroclone-like cell (in the case of PSC-derived cultured). In one embodiment, the colonies are derived from a paraclone or a paraclone-like cell (in the case of PSC-derived cultured). In one embodiment, a majority of the colonies are derived from a holoclone or a holoclone-like cell (in the case of PSC-derived cultured) and/or a meroclone or a meroclone-like cell (in the case of PSC-derived cultured).

In one embodiment, the colony forming efficiency of (the one or more holoclones and/or meroclones among) the differentiated population of PSCs (e.g. among the cells of a dissociated skin organoid, such as a hair-bearing skin organoid) is greater than 1%. In one embodiment, the colony forming efficiency of (the one or more holoclones and/or meroclones among) the differentiated population of PSCs (e.g. among the cells of a dissociated skin organoid, such as a hair-bearing skin organoid) is greater than 2%. In one embodiment, the colony forming efficiency of (the one or more holoclones and/or meroclones among) the differentiated population of PSCs (e.g. among the cells of a dissociated skin organoid, such as a hair-bearing skin organoid) is greater than 3%. In one embodiment, the colony forming efficiency of (the one or more holoclones and/or meroclones among) the differentiated population of PSCs (e.g. among the cells of a dissociated skin organoid, such as a hair-bearing skin organoid) is greater than 4%. In one embodiment, the colony forming efficiency of (the one or more holoclones and/or meroclones among) the differentiated population of PSCs (e.g. among the cells of a dissociated skin organoid, such as a hair-bearing skin organoid) is greater than 5%. In one embodiment, the colony forming efficiency of (the one or more holoclones and/or meroclones among) the differentiated population of PSCs (e.g. among the cells of a dissociated skin organoid, such as a hair-bearing skin organoid) is greater than 6%. In one embodiment, the colony forming efficiency of (the one or more holoclones and/or meroclones among) the differentiated population of PSCs (e.g. among the cells of a dissociated skin organoid, such as a hair-bearing skin organoid) is greater than 7%. In one embodiment, the colony forming efficiency of (the one or more holoclones and/or meroclones among) the differentiated population of PSCs (e.g. among the cells of a dissociated skin organoid, such as a hair-bearing skin organoid) is greater than 8%. In one embodiment, the colony forming efficiency of (the one or more holoclones and/or meroclones among) the differentiated population of PSCs (e.g. among the cells of a dissociated skin organoid, such as a hair-bearing skin organoid) is greater than 9%. In one embodiment, the colony forming efficiency of (the one or more holoclones and/or meroclones among) the differentiated population of PSCs (e.g. among the cells of a dissociated skin organoid, such as a hair-bearing skin organoid) is 10% or greater.

In one embodiment, the methods further comprise yielding more (epidermal) keratinocytes than contaminating (e.g. off-target) cell types when the contacting and the culturing steps are performed using media of this disclosure, in comparison to using basal medium not supplemented with one or more, two or more, or each of the inhibitor of TGFB signaling, the gamma secretase inhibitor, and the agent that disrupts cytoskeletal structure. Thus, preferential expansion/generation/enrichment of keratinocytes, such as epidermal keratinocytes, may be advantageously and surprisingly achieved using media and methods as disclosed herein, relative to other commercial media.

Efficiency of the media and methods disclosed herein to generate/expand/enrich for keratinocytes, such as epidermal keratinocytes may be assessed by interrogating biomarkers. Markers of keratinocytes, such as epidermal keratinocytes, may include basal layer markers such as Keratin 14 (K14), Keratin 5 (K5), Keratin 15 (K15), TP63, Integrin alpha 6 (ITGA6), Integrin beta 1 (ITGB1), and Collagen XVII (BP180). Efficiency may also be assessed by interrogating separately or in parallel biomarkers that should not be expressed at appreciable levels among (epidermal) keratinocytes, including suprabasal layer markers such as Keratin 10 (K10) and/or Involucrin.

The methods may further comprise differentiating the keratinocytes, such as epidermal keratinocytes, that are generated/enriched/expanded using the media or according to the methods disclosed herein. Keratinocytes, such as epidermal keratinocytes, may be differentiated using any method or media known by the person skilled in the art. In one embodiment, the (epidermal) keratinocytes may be differentiated in the presence of a solution comprising a high calcium concentration, such as >1 mM CaCl₂). In one embodiment, the (epidermal) keratinocytes may be differentiated in air-liquid interface conditions. When differentiated, whether in ALI conditions or otherwise, primary keratinocytes or PSC-derived keratinocytes should be able to generate stratified epithelial cells with the appropriate cellular architecture and marker expression. In the case of PSC-derived keratinocytes, such appropriate architecture and marker expression should be consistent, similar, or comparable to their in vivo counterparts.

In one embodiment, keratinocytes, such as epidermal keratinocytes, that are generated and/or expanded and/or enriched using the media and/or methods of this disclosure may be used for therapeutic or cosmetic purposes. In one embodiment, such keratinocytes may be combined with an appropriate scaffold to generate an in vitro-derived skin graft. In one embodiment, such keratinocytes may be used to study the development of skin, and or the biology of various skin-related diseases. In one embodiment, such keratinocytes or in vitro-derived skin grafts may be used to test compounds or other substances that are intended for use on the skin of subjects.

The following non-limiting examples are illustrative of the present disclosure.

### EXAMPLES

### Example 1: Maintaining Pluripotent Stem Cells

Pluripotent stem cells (PSCs) were maintained in PSC maintenance media (mTeSR1™, TeSR™-E8™, or mTeSR™ Plus) (STEMCELL Technologies) and passaged in accordance with the manufacturer's recommended maintenance culture and passaging protocols. At the outset of a keratinocyte derivation protocol, between 1000-3500 PSCs were aggregated in each microwell of an AggreWell™ 800 (STEMCELL Technologies) microwell device or per well of a low-attachment 96-well plate, and cultured for 2 days in the same medium used to maintain the PSCs, in this case one of mTeSR1™, TeSR™-E8™, or mTeSR™ Plus (STEMCELL Technologies) to form aggregates.

### Example 2: Forming Skin Organoids

The aggregates of Example 1 were plated under non-adherent conditions in an AggreWell™ or a 96-well plate, and cultured for 12 days in a skin organoid induction medium, such as STEMdiff™ Skin Organoid Induction Medium (STEMCELL Technologies). Following, the skin organoids are transferred into STEMdiff™ Skin Organoids Maturation Medium (STEMCELL Technologies) for at least 8 weeks, adding or changing the medium every 3-4 days. After the skin organoids have matured, preferably presenting as hair-bearing skin organoids (e.g. hair placode present skin organoids), they are dissociated overnight in a 37 degree incubator in a Dispase solution.

### Example 3: Plating Dissociated Cells of Skin Organoids Under Adherent Conditions

Dissociated organoids of Example 2 are plated at an appropriate density, usually ranging between one dissociated organoid per well of a 6-well plate up to a T25 flask, in a cell culture medium of this disclosure (e.g. in DermaCult™, STEMCELL Technologies). Once large colonies appeared they were passaged at or just prior to confluency using Accutase™ or a trypsin-based dissociation reagent. After transferring the cells into DermaCult™ medium, the cultures are passaged according to the manufacturer's recommended protocol where 1.25-5×10³ cells are seeded per cm².

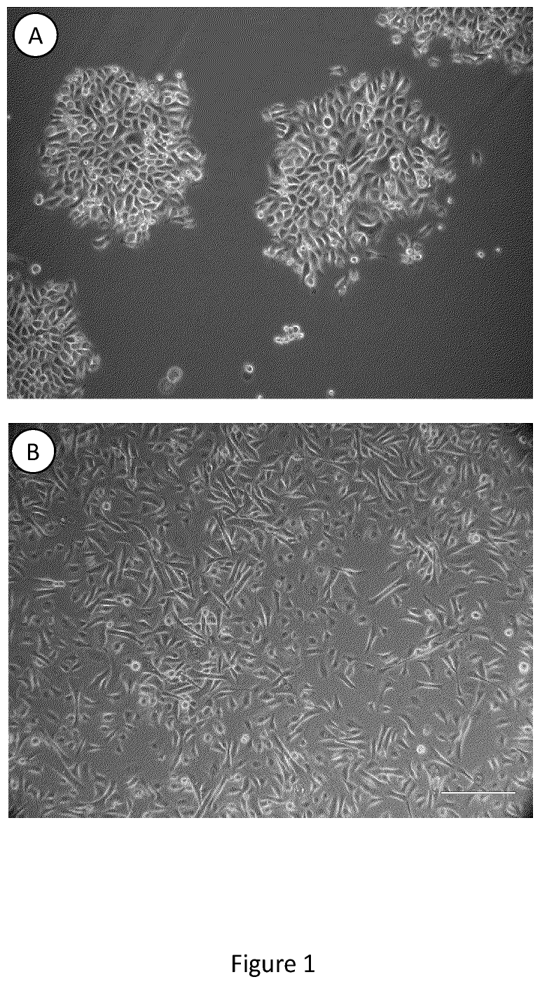

Primary human keratinocytes in culture exhibit a cobblestone epithelial cell morphology (FIG. 1A). Also, primary human keratinocytes should express basal layer markers K14, K5, p63, ITGA6, and ITGB1, but should not express marked levels of suprabasal layer marker K10 and involucrin. However, when PSC are directly seeded into monolayer conditions in a keratinocyte medium and differentiated using a published protocol (Kajiwara K, et al, 2017), the morphology of the arising cells is not cobblestone-like. Rather, the arising cells appear to be elongated (as fibroblasts) and scattered and overlapping (FIG. 1B). Briefly, the Kajiwara K, et al, 2017 protocol involves culturing PSC for 4 days in Defined Keratinocyte Serum Free Medium (“DKSFM”) (Thermo Fisher) supplemented with Retinoic Acid and BMP4 on Vitronectin coated plates. From days 4-14, the cells are cultured in DKSFM supplemented with 20 ng/ml EGF. After 14 days, the cells are cultured in DKSFM supplemented with 20 ng/ml EGF and 10 uM Y-27632 on Fibronectin and Type 1 collagen coated plates.

Thus, an improved means of generating PSC-derived keratinocytes is needed, and the foregoing observation prompted the experiments that led to the unexpected discovery that cells of dissociated skin organoids seem to provide a surprisingly effective starting point for expanding relatively pure cultures of PSC-derived keratinocytes.

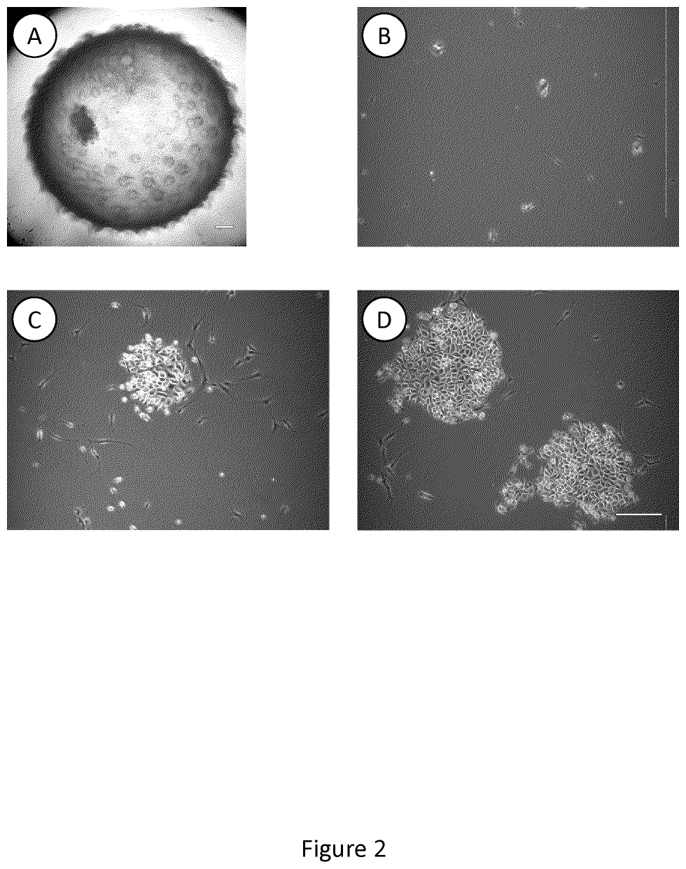

Skin organoids may be generated from PSC using STEMdiff™ Skin Organoids Induction Medium (STEMCELL Technologies). FIG. 2A shows a representative image of a hair-bearing skin organoid formed using STEMdiff™ Skin Organoid Induction Medium (STEMCELL Technologies), formed as described in Examples 1 and 2. The skin organoids were initiated in the foregoing medium in accordance with the manufacturer's recommended protocol, and after 12 days the induced skin organoids were transferred into STEMdiff™ Skin Organoid Maturation Medium (STEMCELL Technologies). Skin organoids were matured under non-adherent conditions with cell culture media changes every 3 to 4 days, for about 6 weeks or longer. Skin organoids may be considered mature when hair follicles are observed on their outer surface pointing toward the cell culture medium.

Once the (hair-bearing) skin organoids have sufficiently matured, they may be incubated in Dispase™ (STEMCELL Technologies) at 37° C. overnight to dissociate the structure into a suspension of cells. The dissociated suspension of cells is plated into a tissue culture treated culture plate or flask, and cultured in DermaCult™ (STEMCELL Technologies) under adherent conditions. The medium was changed about every 2-3 days, and before the cells reached confluency they were passaged using Accutase or a trypsin-based dissociation reagent. After initially seeding the differentiated population of PSCs, few colonies of keratinocytes are observed at day 3 or 4 (FIG. 2B). However, after about 7 days in culture large colonies of keratinocytes emerge (FIGS. 2C and 2D).

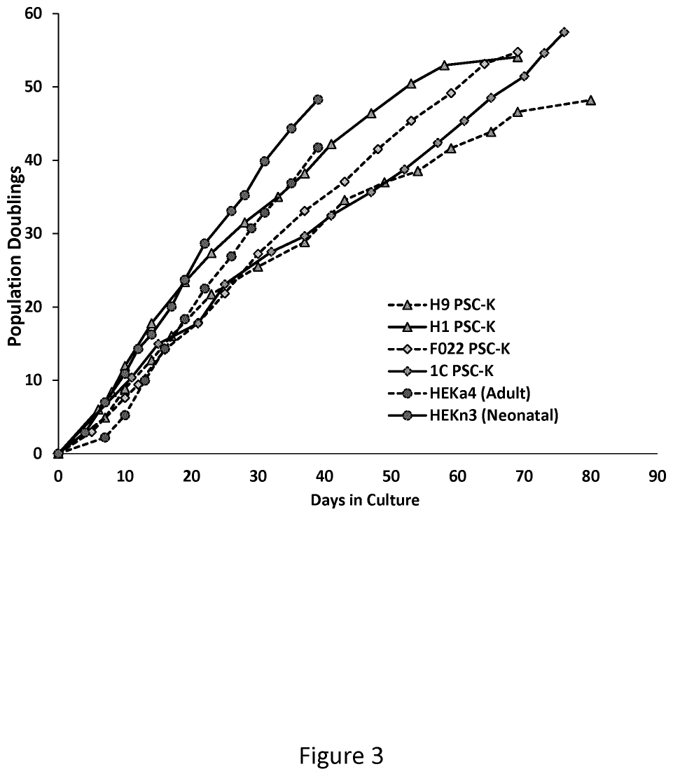

Population doublings were measured over the course of several weeks for keratinocytes derived in accordance with this Example 3 from three different human PSC lines (H9 ES cells, H1 ES cells, and STiPS F022 iPS cells). The expansion of the human PSC-derived keratinocytes was compared against commercially sourced primary adult human keratinocytes and primary neonatal human keratinocytes. FIG. 3 shows that over the course of the experiment population doublings of the PSC-derived keratinocytes was only slightly decreased and thus comparable to the primary keratinocyte controls.

### Example 4: Expansion of PSC-Derived Keratinocytes is Increased when Cells are Seeded on Collagen-Coated Plates

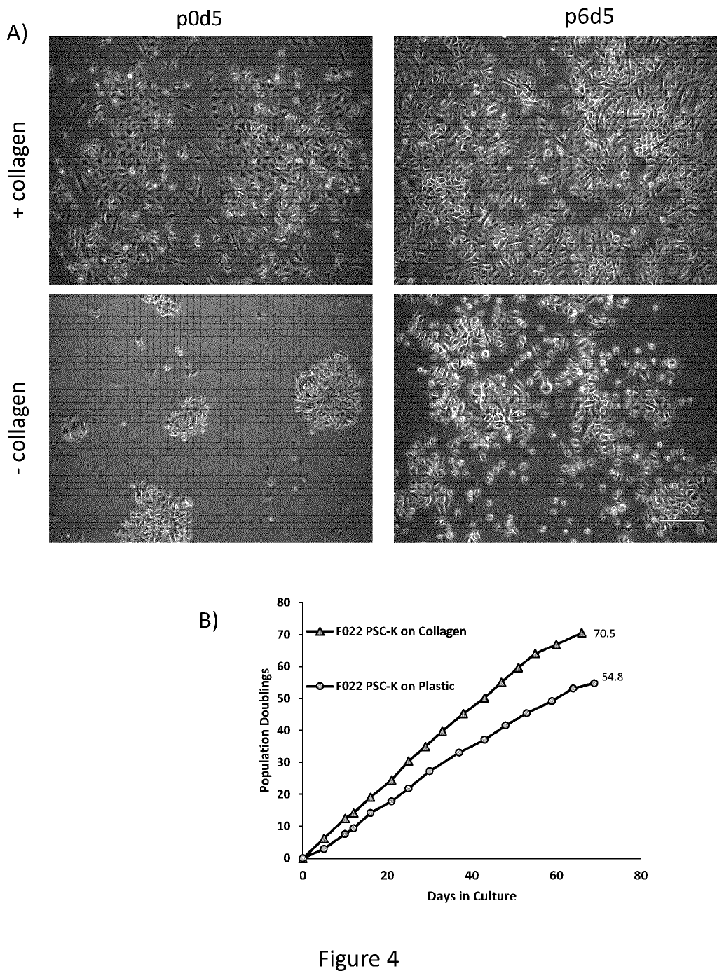

The effect of extracellular matrices on population doubling of PSC-derived keratinocytes was explored. Keratinocytes derived from F022 iPS cells were formed and plated essentially as described in Example 3, except the cell suspension of dissociated skin organoids were plated either directly on plastic dishes or on dishes coated with collagen (STEMCELL Technologies). At both an early passage (passage 0) and at a later passage (passage 6), there appeared to be more PSC-derived keratinocytes when grown on the collagen coated surface (FIG. 4A). This observation was confirmed when the number of PSC-derived keratinocytes was quantified over the course of several weeks in the foregoing culture conditions. Culturing a cell suspension of dissociated skin organoids onto collagen-coated plates increased the number of population doublings by approximately ten, in comparison to the condition where the cells were plated directly onto plastic (FIG. 4B).

### Example 5: PSC-Derived Keratinocytes Exhibit Comparable Levels of Basal Cells Markers in Comparison to a Control Culture of Primary Keratinocytes

PSC-derived keratinocytes generated in accordance with Examples 3 or 4 were assessed for the expression of keratinocyte markers by immunocytochemistry. Briefly, samples were fixed in 4% PFA for 1 hour at room temperature or overnight at 4 degrees. Samples were then exposed to blocking buffer for 1 hour at room temperature. Following, the samples were incubated with primary antibodies (as exemplified in the table below) in diluted PBST overnight at 4 degrees Celsius. After a series of washes, the samples were incubated with secondary antibodies diluted in PBST for 1 hour at room temperature in the dark. After a series of washes, the samples were incubated with DAPI stain (1:10000 in PBS) for 8 minutes at room temperature. A series of washes using PBS were done before the samples were visualized using fluorescence microscopy.

| Protein of | | | | Recommended |
|---|---|---|---|---|
| Interest | Supplier | Catalog No | Species | Dilution |
| Keratin 14 | Biolegend | 905304 | Rabbit | 1:2000 |
| p63 | Abcam | ab124762 | Rabbit | 1:600 |
| Keratin 5 | Biolegend | 905501 | Rabbit | 1:800 |
| Keratin 10 | Abcam | ab9026 | Mouse | 1:1000 |
| Involucrin | Abcam | ab53112 | Rabbit | 1:100 |
| Loricrin | Abcam | ab85679 | Rabbit | 1:200 |

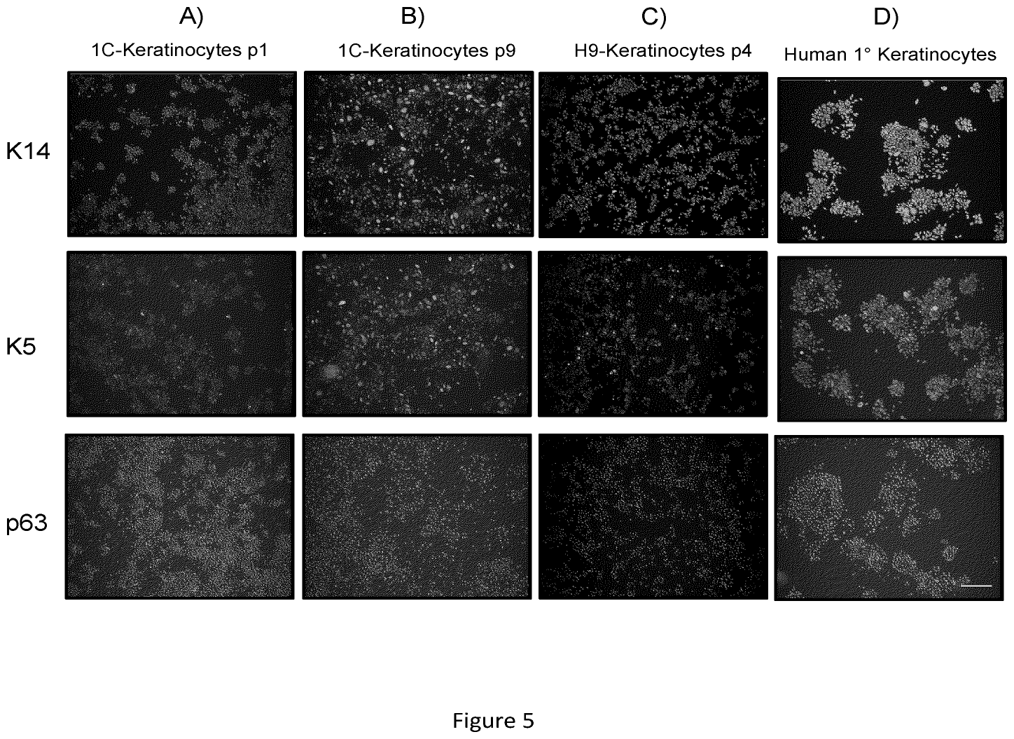

The expression of Keratin 14 (K14), Keratin 5 (K5), and TP63 (p63) was confirmed in WLS-1C iPS cells at passage 1 and passage 9 (FIGS. 5A and 5B) and in H9 ES cells at passage 4 (FIG. 5C). The expression levels of these markers appeared comparable to the expression of these markers among a culture of human primary keratinocytes (FIG. 5D). These results indicate that the PSC-derived keratinocytes bear good resemblance to primary keratinocytes.

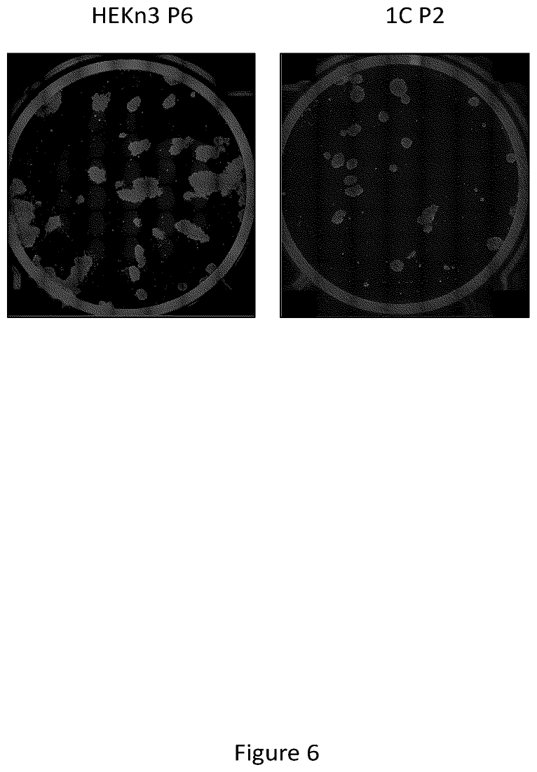

To test the capacity of the dissociated cells of a PSC-derived skin organoid to form colonies, limiting numbers of primary keratinocytes and PSC-derived cells (200 total) were plated into a well of a 6-well plate in DermaCult™ but in the absence of any coating or feeder layer (FIG. 6). The colonies that emerged were stained for K14, and it was apparent that both types of starting cell populations possessed cells capable of forming K14+ colonies. An apparent colony forming efficiency of greater than 5%, and likely about 10%, was achieved. Considering the morphology and the colony sizes it was apparent that both starting cell populations included numerous holoclones and meroclones among the 200 seeded cells. These data suggest that the media and methods of this disclosure support the expansion and clonal derivation of keratinocyte stem cells or early keratinocyte progenitor cells.

### Example 6: Analysis of Cells Grown Using Commercially Available Media

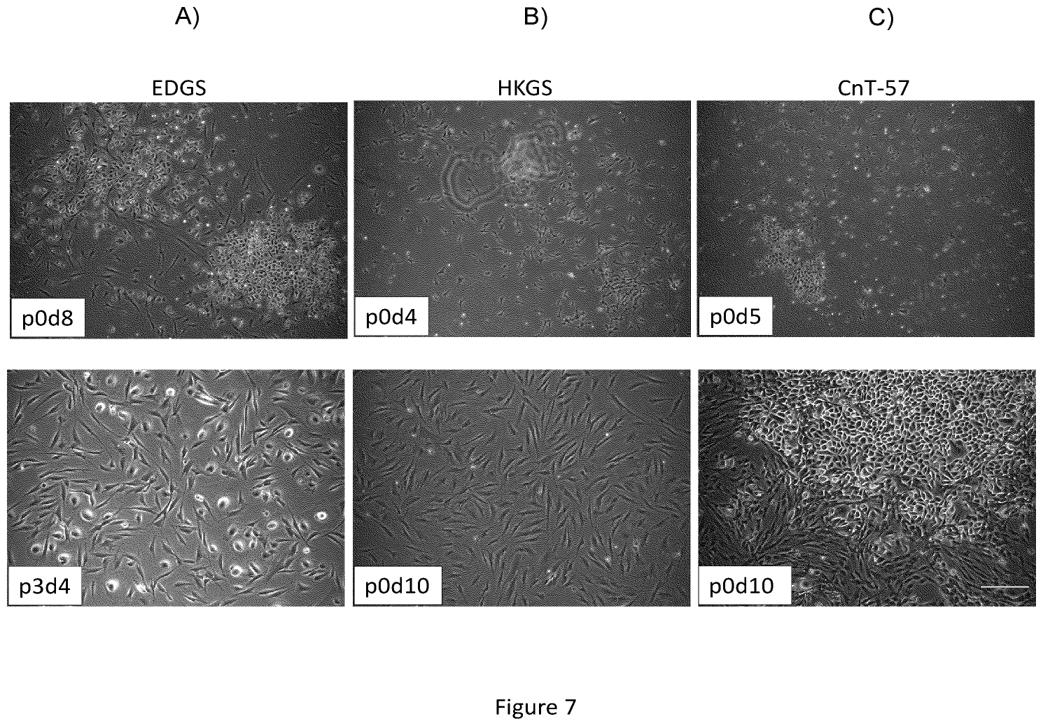

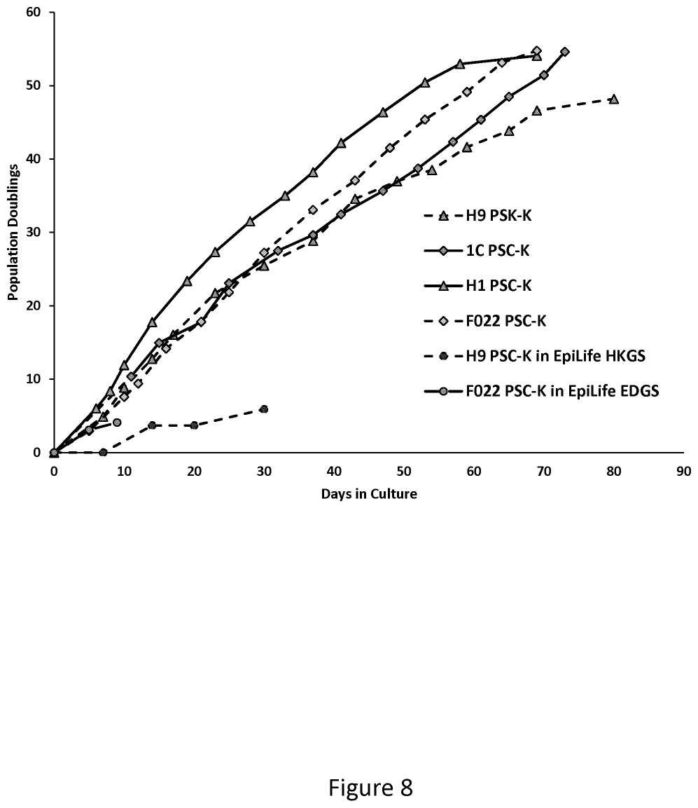

Various commercially available cell culture media are marketed for the purpose of culturing human epidermal keratinocytes: EpiLife™ Defined Growth Supplement for use with EpiLife™ medium; Gibco™ Human Keratinocyte Growth Supplement for use with EpiLife medium or Medium 154; and Cnt-57 Epithelial Proliferation Medium. These media were tested in experiments to generate an expanded population of PSC-derived epidermal keratinocytes, starting from a cell suspension of dissociated skin organoids obtained essentially as described in Example 3. Images of the arising cells taken at different time points after culturing in either EpiLife EDGS (FIG. 7A), EpiLife HKGS (FIG. 7B) and CnT-57 (FIG. 6C) show that these media are not capable of expanding PSC-derived keratinocytes that exhibit a cobblestone morphology. Population doublings were also assessed over several weeks for keratinocytes grown from various PSC lines in a culture medium of this disclosure in comparison to EpiLife EDGS and EpiLife HKGS. DermaCult™ outperformed both EpiLife EDGS and EpiLife HKGS for all cell lines tested (FIG. 8). These results suggest that EpiLife EDGS, EpiLife HKGS, and CnT-57 are not capable of generating an expanded population of PSC-derived keratinocytes.

Further experimentation was conducted using DermaCult™ and the commercially available media described above wherein the expression of Keratin-14 and TP63 was assessed among keratinocytes differentiated from different PSC lines.

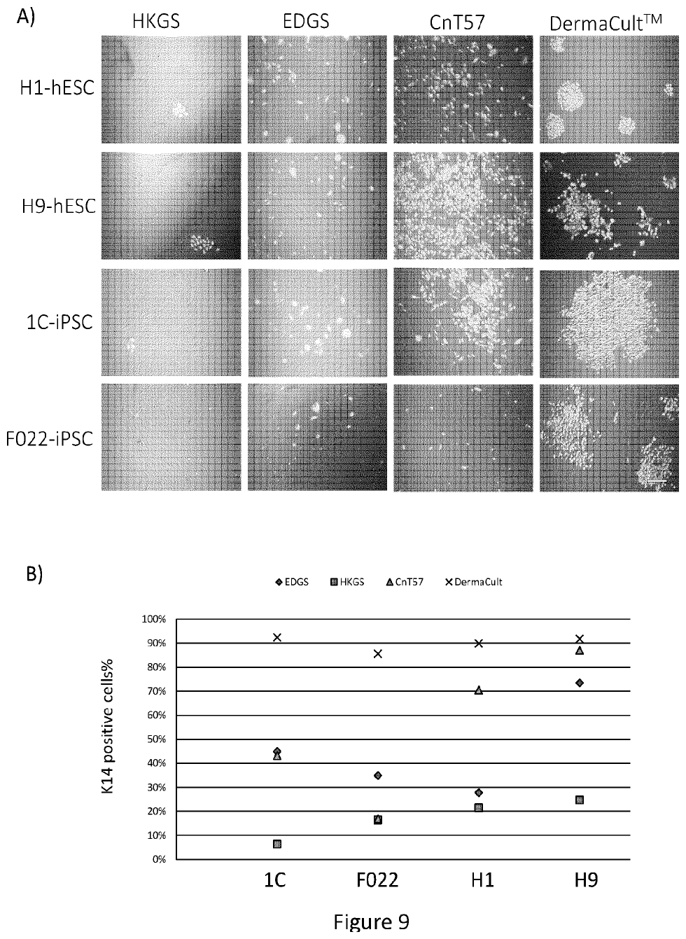

FIG. 9A shows representative images of Keratin 14 expression among cells differentiated from two ES cell lines (H1 and H9) and two iPS cell lines (WLS-1C and STiPS F022) in either EpiLife HKGS, EpiLife EDGS, CnT-57, and DermaCult™. While the images suggested the DermaCult™ formulation outperformed the commercial formulations, the output PSC-derived keratinocytes were quantified to confirm these observations (FIG. 9B). The cells were quantified using Image J and there were clearly more K14+ cells generated in DermaCult™ in comparison to the commercial media tested. Notably, the percentage of K14+ cells generated using DermaCult™ was consistent across keratinocytes generated from each of the PSC lines tested.

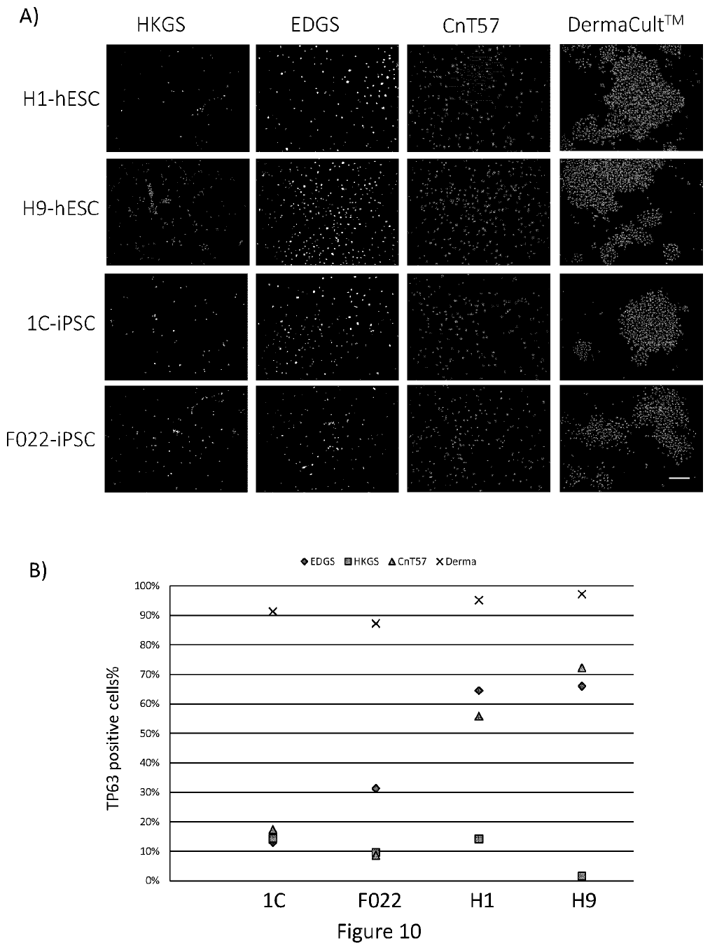

FIG. 10A shows representative images of TP63 expression among keratinocytes differentiated from two ES cell lines (H1 and H9) and two iPS cell lines (WLS-1C and STiPS F022) in either EpiLife HKGS, EpiLife EDGS, CnT-57, and DermaCult™. While the images suggested the DermaCult™ formulation outperformed the commercial formulations, the output PSC-derived keratinocytes were quantified to confirm these observations (FIG. 10B). The cells were quantified as described above in respect of FIG. 9 and there were clearly more TP63+ cells generated in DermaCult™ in comparison to the commercial media tested. Notably, the percentage of TP63+ cells generated using DermaCult™ was consistent across keratinocytes generated from each of the PSC lines tested.

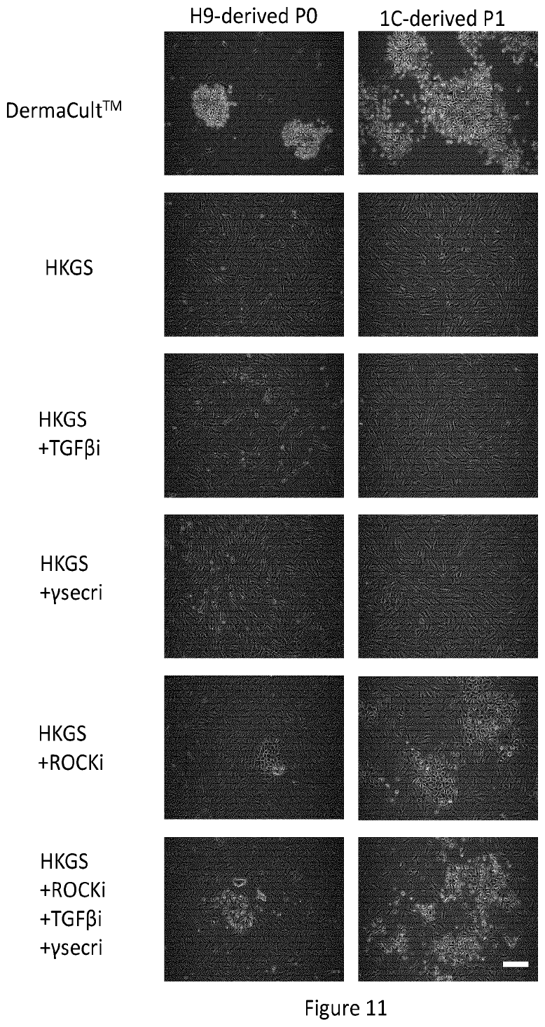

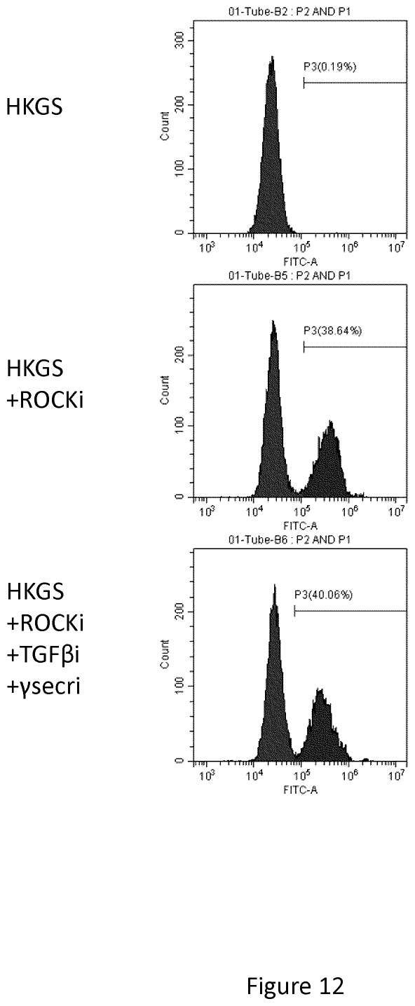

The ability of a commercially available medium to expand PSC-derived keratinocytes could be somewhat restored upon supplementation with one or more small molecule inhibitors, but not to the levels of DermaCult™ (FIG. 11). Specifically, HKGS medium was supplemented with A83-01, DAPT, Y-27632, or all three of A83-01, DAPT, and Y-27632. FIG. 10 shows the emergence of colonies when an ES cell line (H9) and an iPS cell line (1C) were cultured in HKGS supplemented with either Y-27632 or with all three of the foregoing factors. These findings were confirmed in a different experiment where 1C iPS cells were cultured in the foregoing media conditions and analyzed for p63 expression, to distinguish keratinocytes from other cell types included in the PSC-derived skin organoids, such as fibroblasts (FIG. 12). Exposing the cells of a dissociated (hair-bearing) skin organoid to either Y-27632 or to all three of the foregoing factors resulted in the emergence of comparable numbers of p63+ cells, in comparison to an absence of emerged p63+ cells after culture in the presence of only HKGS.

1. A method of expanding epidermal keratinocytes, the method comprising:
contacting a differentiated population of pluripotent stem cells (PSCs) with an expansion medium comprising a basal medium and one or more of an inhibitor of transformation growth factor (TGF) signaling, a gamma secretase inhibitor, and an agent that disrupts cytoskeletal structure; and
culturing the differentiated population of PSCs in the expansion medium to generate expanded epidermal keratinocytes.

2. The method according to claim 1, wherein the expansion medium comprises two or more of the inhibitor of TGF signaling, the gamma secretase inhibitor, and the agent that disrupts cytoskeletal structure.

3. The method according to claim 1, wherein the expansion medium is serum- and/or bovine pituitary extract-free.

4. The method according to claim 1, wherein the contacting and the culturing steps are;
(i) in feeder cell free conditions; and/or
(ii) performed on or in a support or coating comprising one or more extracellular matrix proteins.

5. (canceled)

6. The method according to claim 1, further comprising dissociating a PSC-derived skin organoid to obtain the differentiated population of PSCs.

7. The method according to claim 6, wherein the PSC-derived skin organoid is:
(i) formed under serum- and or feeder cell-free conditions; and/or
(ii) a PSC-derived hair-bearing skin organoid.

8. (canceled)

9. The method according to claim 1, further comprising yielding more epidermal keratinocytes than contaminating cell types when the contacting and the culturing steps are performed in the expansion medium compared to a basal medium not supplemented with one or more of the inhibitor of TGFB signaling, the gamma secretase inhibitor, and the agent that disrupts cytoskeletal structure.

10. The method according to claim 1, wherein the inhibitor of TGF signaling is;
(i) an inhibitor of TGF-beta signaling; and/or
(ii) one or more of A83-01, A77-01, and SB431542.

11. (canceled)

12. The method according to claim 1, wherein the gamma secretase inhibitor is DAPT.

13. The method according to claim 1, wherein the agent that disrupts cytoskeletal structure is a Rho/Rock kinase inhibitor.

14. The method according to claim 13, wherein the Rho/Rock kinase inhibitor is Y-27632.

15. An epidermal keratinocyte expansion medium, comprising a basal medium supplemented with one or more of an inhibitor of transformation growth factor (TGF) signaling, a gamma secretase inhibitor, and an agent that disrupts cytoskeletal structure.

16. The medium according to claim 15, wherein the inhibitor of TGF signaling is an inhibitor of TGF-beta signaling.

17. The medium according to claim 15, wherein the inhibitor of TGF-beta signaling is one or more of A83-01, A77-01, and SB431542.

18. The medium according to claim 15, wherein the gamma secretase inhibitor is DAPT.

19. The medium according to claim 15, wherein the agent that disrupts cytoskeletal structure is a Rho/Rock kinase inhibitor.

20. The medium according to claim 19, wherein the Rho/Rock kinase inhibitor is Y-27632.

21. The medium according to claim 15, wherein the medium does not come into contact with feeder cells to expand epidermal keratinocytes.

22. (canceled)

23. The medium according to claim 15, wherein the medium is serum- and/or BPE-free.

24. The medium according to any one claim 15, wherein the medium is free of:
(i) an exogenously added inhibitor of TGF signaling; and/or
(ii) an exogenously added gamma secretase inhibitor.

25. (canceled)

26. (canceled)
